(19) United States
(12) Patent Application Publication
(10) Pub. No.: US 2012/0070878 A1
(43) Pub. Date: Mar. 22, 2012

(54) Microporous Microfluidic Device

(76) Inventors: Katherine A. Fink, Campbell, NY (US); Vasiliy Nikolaevich Goral, Painted Post, NY (US); Hui Su, Big Flats, NY (US); Po Ki Yuen, Painted Post, NY (US)

(21) Appl. No.: 13/234,490

(22) Filed: Sep. 16, 2011

Related U.S. Application Data
(60) Provisional application No. 61/385,213, filed on Sep. 22, 2010.

(51) Int. Cl. C12N 1/00; C08J 9/26; B01J 19/00; C12M 1/00; C01B 31/00

(52) U.S. Cl. 435/243; 435/289.1; 423/437.1; 422/162; 264/49

(57) ABSTRACT

A micro fluidic apparatus includes (i) a first conduit; (ii) a second conduit; and (iii) a first interconnected microporous network in communication with the first and second conduits and configured to allow diffusion of gas between the first and second conduits. The microporous network comprises poly(dimethylsiloxane) (PDMS) and prevents flow of aqueous fluid between the first and second conduits through the microporous network.

### CROSS-REFERENCE

This application claims the benefit of priority under 35 U.S.C. §119 of U.S. Provisional Application Ser. No. 61/385213 filed on Sep. 22, 2010 the content of which is relied upon and incorporated herein by reference in its entirety.

### FIELD

The present disclosure relates to microfluidic devices having interconnected microporous structures, particularly to micro fluidic devices formed at least in part by poly(dimethylsiloxane) (PDMS).

### BACKGROUND

Microfluidics is emerging as one of the fastest growing fields for chemical and biological applications, and a good deal of effort has been expended in identifying suitable materials and novel functional attributes for use in microfluidic devices. One attractive feature that can be incorporated into micro fluidic devices is a porous membrane or porous regions or materials within or between microfluidic channels. Such porous regions may allow for selective diffusion of gases or other chemical species from one microfluidic channel to another and can have a variety of potential uses, including multiphase catalytic reactions in chemical and pharmaceutical applications. Because of the wide variety of uses and the rapidly growing field, demand has increased for methods of rapidly fabricating low-cost microfluidic devices.

PDMS microfluidic devices fabricated by soft lithography have been widely used for various chemical and biological applications since they were first reported in the late 1990s. The use of PDMS within microfluidic devices allows ease of fabrication, rapid prototyping and reduced material costs. To date, no PDMS microfluidic device having microporous regions have been described. Perhaps this is because microporous PDMS tends to be optically opaque, which would make it difficult to observe microfluidic channels of the device.

### BRIEF SUMMARY

The present disclosure describes, among other things, microfluidic devices having microporous PDMS regions and an optically transparent portion that allows for viewing of the channels. Such microfluidic devices have the benefits of PDMS devices, which include ease of fabrication, rapid prototyping and reduced material costs, and the benefits of microporous devices, while still allowing visual observation of desired parts of the device while in use. In addition due to the hydrophobic nature of microporous PDMS, aqueous liquids but not gasses are prevented from passing through the microporous PDMS from one microfluidic conduit to another. Thus, selective diffusion of gasses can advantageously be achieved.

In various embodiments described herein, a microfluidic device includes a first conduit, a second conduit; and a three-dimensional (3D) interconnected microporous network in communication with the first and second conduits. The microporous network comprises PDMS and prevents flow of aqueous fluid between the first and second conduits through the microporous network, but is configured (via its interconnected microporous network) to allow diffusion of gas between the first and second conduits.

In numerous embodiments, a method for manufacturing a microfluidic device includes disposing a composition comprising a PDMS prepolymer and a porogen in a mold. The mold configured to form a first part of the microfluidic device. The first part of the device has first and second channels. The method further includes curing the composition in the mold to form the first part. The material forming the first part comprises a PDMS polymer interspersed with the porogen. The method also includes removing the porogen from the PDMS polymer to generate a porous first part comprising a porous PDMS polymer. In addition, the method includes sealing the porous first part to a second part such that a surface of the second part and the first and second channels of the first part together form first and second conduits of the microfluidic device.

In some embodiments, a method for manufacturing a microfluidic device includes disposing a composition comprising a PDMS prepolymer in a mold. The mold is configured to form a first part of the microfluidic device. The first part of the device has first and second channels. The method also includes curing the composition in the mold to form the first part. The method further includes providing a second part of the device. The second part contains PDMS having an interconnected microporous structure. In addition, the method includes sealing the first part to a second part such that a surface of the second part and the first and second channels of the first part together form first and second conduits of the microfluidic device.

The devices and methods described herein may provide one or more advantages over prior microfluidic devices and methods. Using the methods described herein, a large range of pore sizes can be fabricated and tuned to a desired range for particular uses. Additionally, a highly interconnected microporous structure with different pore sizes can be fabricated in a single device by using different sizes of porogen in the pre-polymer before curing. Further, there is essentially no limitation on the physical size of the molded 3D interconnected microporous structures. Further, microporous microfluidic device in 3D configurations can be fabricated. Also, gas transportation efficiency through the 3D interconnected microporous structures is controllable because of large range of pore sizes can be used to fabricate the device. Additionally, device assembly can be performed efficiently, using simple processes. These and other advantages of the various embodiments of the devices and methods described herein will be readily apparent to those of skill in the art upon reading the disclosure presented herein.

### BRIEF DESCRIPTION OF THE DRAWINGS

FIG. 1 is a schematic exploded perspective view of a microfluidic device having two parts.

FIG. 2 is a schematic perspective view of the microfluidic device of FIG. 1 in assembled form.

FIG. 3 is a schematic cross-sectional view of the microfluidic device of FIG. 2.

FIGS. 4-6 are schematic cross-sectional views of embodiments of microfluidic devices.

FIGS. 7-8 are flow diagrams illustrating overviews of embodiments of methods described herein.

FIG. 9A is an image of a microstructured mold fabricated from three layers of custom cut self-adhesive white vinyl sheet adhered to a glass slide.

FIG. 9B is a fully assembled microporous PDMS microfluidic device fabricated using the mold depicted in FIG. 9A.

FIGS. 10A-D are top views (A and C) and cross-sectional views (B and D) of scanning electron microscope images of microporous PDMS structures fabricated from 150 micrometer to 180 micrometer sugar particles. (C) and (D) are zoomed in views of the dashed line area of (A) and (B), respectively.

FIG. 11 is an image of water droplets on microporous PDMS substrates fabricated by various sizes of pre-sieved sugar particles.

FIGS. 12A-D are time lapse images of microfluidic devices showing acidification of water by a CO₂ gas experiment at 0 sec. (A), 30 sec. (B), 50 sec. (C), and 1 min. 30 sec. (D).

FIGS. 13A-D are time lapse images of microfluidic devices showing acidification of water by a CO₂ gas experiment at 0 sec. (A), 45 sec. (B), 1 min. 30 sec. (C), and 4 min. 30 sec. (D).

The schematic drawings presented herein are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

### DETAILED DESCRIPTION

In the following detailed description, reference is made to the accompanying drawings that form a part hereof, and in which are shown by way of illustration several specific embodiments of devices, systems and methods. It is to be understood that other embodiments are contemplated and may be made without departing from the scope or spirit of the present disclosure. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise.

As used in this specification and the appended claims, the term “or” is generally employed in its sense including “and/or” unless the content clearly dictates otherwise.

As used herein, “have”, “having”, “include”, “including”, “comprise”, “comprising” or the like are used in their open ended sense, and generally mean “including, but not limited to.” It will be understood that the terms “consisting of” and “consisting essentially of” are subsumed in the term “comprising.” For example, a part of a microfluidic device comprising a microporous PDMS polymer may consist of, or consist essentially of, the microporous PDMS polymer

“Consisting essentially of”, as it relates to a compositions, articles, systems, apparatuses or methods, means that the compositions, articles, systems, apparatuses or methods include only the recited components or steps of the compositions, articles, systems, apparatuses or methods and, optionally, other components or steps that do not materially affect the basic and novel properties of the compositions, articles, systems, apparatuses or methods.

Any direction referred to herein, such as “top,” “bottom,” “left,” “right,” “upper,” “lower,” “above,” below,” and other directions and orientations are described herein for clarity in reference to the figures and are not to be limiting of an actual device or system or use of the device or system. Devices or systems as described herein may be used in a number of directions and orientations.

As used herein, “interconnected microporous structure” refers to a structure having pores or interstices of an average diametric size of less than 1000 micrometers in which pores or interstices are interconnected such that fluid (e.g., liquid, gas, or vapor) may travel between pores or interstices from one surface of the structure to another surface of the structure. It will be understood that interconnected microporous structures may have some “dead ends” or “no-outlets” or “isolated voids.”

As used herein, “pore” means a cavity or void in a surface, a body, or both a surface and a body of a solid article, where the cavity or void has at least one outer opening at a surface of the article.

As used herein, “interstice” means a cavity or void in a body of a solid polymer not having a direct outer opening at a surface of the article, i.e., not a pore, but may have an indirect outer opening or pathway to an outer surface of the article by way of one or more links or connections to adjacent or neighbor “pores” “interstices,” or a combination thereof.

The present disclosure describes, among other things, microfluidic devices having at least a portion formed from microporous PDMS. The microporous PDMS is in communication with a first and second conduit of the device and allows diffusion of gases, vapors and the like between the conduits through the interconnected microporous structure of the PDMS. Due to the hydrophobic nature of the microporous PDMS, aqueous liquids in the first or second conduit will not diffuse to the other conduit through the microporous PDMS. Thus, the microfluidic devices may be advantageously employed in situations where interaction or exchange of gasses between conduits, but not interaction or exchange of aqueous liquids between conduits, is desired. For example, the microfluidic devices may be used for cell culture with the interconnected microporous network providing for rapid exchange of carbon dioxide and oxygen from and to cells; the microfluidic devices may serve as micro-reactors for multiphasic reactions, such as gas-liquid, or gas-liquid-solid reactions; the microfluidic devices may be used for purposes of sample filtration, fluid mixing, valving; or the like.

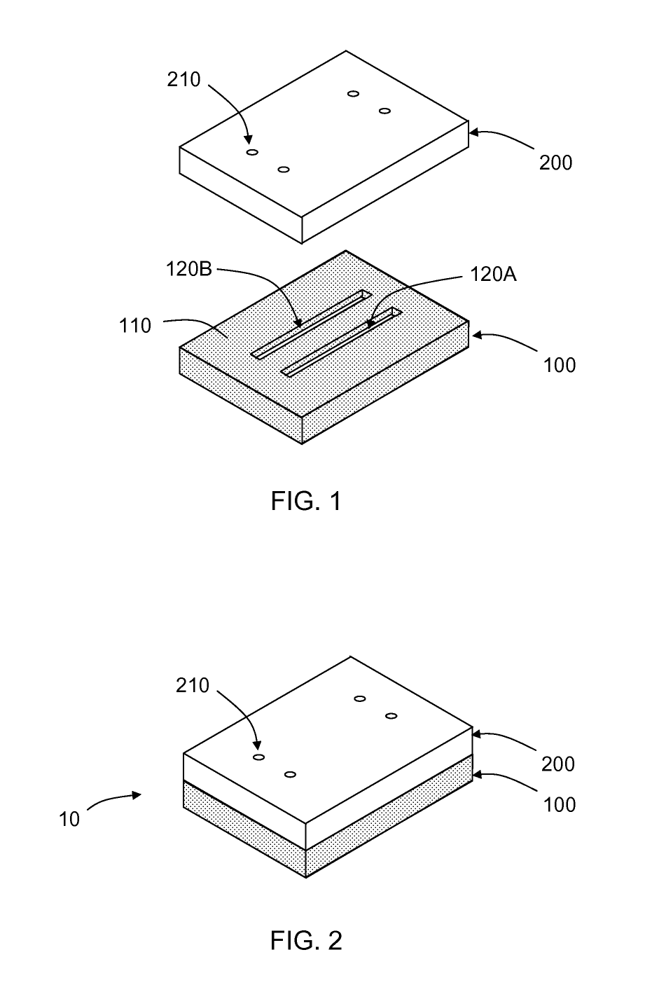

In various embodiments, a microfluidic device is formed from two parts. For example and with references to FIGS. 1-3, the device 10 may be formed from a top part 200 and a bottom part 100. In the device 10 depicted in FIGS. 1-3, the bottom 100 part is formed from microporous PDMS having a 3D interconnected microporous network 110. Channels 120A, 120B are formed in the bottom part 100, and together with a surface 220 of the top part 200, serve to form a part of the fluid conduits 250 of the device when fully assembled.

The top part 200 may be a plate, film, lid, or any other article suitable to cover the channels 120A, 120B of the first part to form the conduits 250A, 250B, 250C. The top part 200 is formed from an optically transparent material and provides an observation window into the conduits 250A, 250B, 250C, which would otherwise not be visible due to the opacity of the interconnected microporous PDMS 110. As used herein, “optically transparent” means an object laying beyond the optically transparent article may be clearly seen. Any suitable optically transparent material or materials may be used to form the optically transparent part 200 that provides the window. For example, the part may be fabricated from inorganic materials such as glass or from plastics or polymers, including dendritic polymers, such as poly(vinyl chloride), poly(vinyl alcohol), poly(methyl methacrylate), poly(vinyl acetate-maleic anhydride), poly(dimethylsiloxane) monomethacrylate, cyclic olefin polymers and copolymers including copolymers of norbornene and ethylene, fluorocarbon polymers, polystyrenes, polypropylene, polyethyleneimine, polycarbonate; copolymers such as poly(vinyl acetate-co-maleic anhydride), poly(styrene-co-maleic anhydride), polysaccharide, polysaccharide peptide, poly(ethylene-co-acrylic acid) or derivatives of these or the like. In some embodiments, the transparent part 200 is formed from non-porous or transparent PDMS or glass substrate.

In embodiments depicted in FIGS. 1-3, the top part 200 is the same or substantially the same length and width as the bottom part 100, and the two parts 100, 200 are aligned. However, it will be understood that the top part 200 (or the part not having the channels 120) may have a length or width that is smaller than that of the bottom part 100, as long as the top part 200 covers the channels 120.

The top part has openings 210 through its depth that serve as inlets or outlets to the conduits 250 when the device 10 is fully assembled. Accordingly, during assembly, the top 200 and bottom 100 parts are aligned so that the openings 210 are aligned with the channels 120A, 120B. Of course, the openings may be formed in the bottom part (or the part having the channels).

Depending on the material used to form the top part 100 or the bottom part 200, the parts may be self sealing. Otherwise, the parts 100, 200 may be adhered or the like to sealingly engage.

Pumps, syringes, or other suitable injection or infusion device may be employed to introduce fluid into an inlet in communication with a conduit of a microfluidic device. The microfluidic devices described herein can readily be adapted for use with available robotic fluid handling systems.

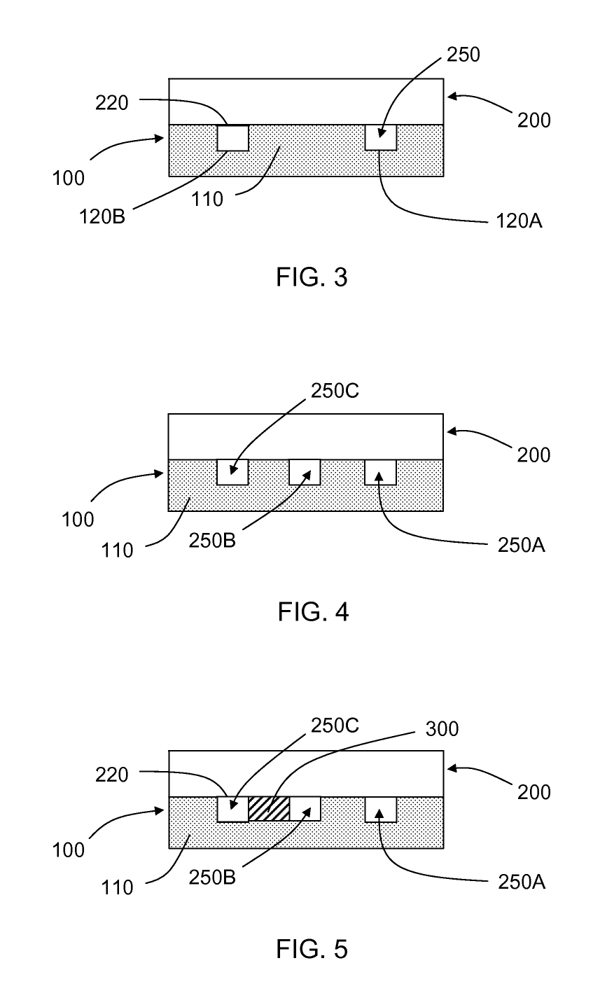

Referring now specifically to FIG. 3, the microfluidic conduits 120A, 120B are in fluid communication with the interconnected microporous network 110 of the microporous PDMS. Thus, gasses, vapors and the like can readily diffuse from one conduit to the other through the microporous network. Because of the hydrophobic nature of microporous PDMS, aqueous fluids do not pass through the microporous network 110. Typically, the microporous PDMS has a water contact angle of 90 degrees or greater (e.g., 100 degrees or greater, 110 degrees or greater, or the like), which is sufficiently hydrophobic to prevent aqueous liquids from passing through the pores.

While it is possible, and may be desirable in some cases, to treat surfaces of the microporous PDMS (e.g., the channels 120A, 120B) to increase the hydrophilicity or wettability, it can be difficult to increase hydrophilicity throughout the microporous network 110. Accordingly, microfluidic devices with more hydrophilic surfaces or conduits may be made, while retaining the hydrophobic nature of the microporous network, and thus preventing aqueous liquids from diffusing through the network. It should be noted that some treatments, such as oxygen plasma treatment, that render surfaces more hydrophilic may not have a lasting effect on PDMS.

Referring now to FIG. 4, a microfluidic device having three microfluidic conduits 250A, 250B, 250C is shown. It will be understood that a microfluidic device may have any suitable and desired number of conduits into which fluid (gas, liquid or the like) may be introduced or from which fluid may be withdrawn. Each conduit may be in communication with an inlet or outlet for introducing or withdrawing fluid from the conduit. As shown in FIG. 4, each conduit 250A, 250B, 250C is in communication with the interconnected microporous network 110 of the microporous PDMS. Thus, gasses, but not aqueous liquids, may pass from one conduit to another through the network 110.

Referring now to FIG. 5, a microfluidic device includes first 110 and second 300 microporous networks. The microporous networks 110, 300 are interconnected and formed from PDMS. The average pore size or the density of pores of the first 110 and second 300 networks is different and allows diffusion of gasses through the networks 110, 300 at different rates. In the depicted embodiment, the second microporous network 300 is in communication with the second 250B and third conduits 250C, while the first interconnected microporous network 110 is in connection with the first 250A, second 250B, and third 250C conduits. By way of example, if the second microporous network 300 is configured to allow more rapid diffusion of gasses (e.g., has larger or higher density of pores) than the first network 110, exchange of gasses between the third 250C and second 250B conduits can occur more rapidly than between the first 250A and second 250B conduits or the first 250A and third 250C conduits.

While the second microporous network 300 is depicted in FIG. 5 as serving as a sidewall and being disposed between the second 250B and third conduits 250C, the network 300 may be in communication with the conduits 250B, 250C in any suitable manner. For example, the network 300 may serve as a bottom or a portion of the sidewall of the conduits 250B, 250C. In some embodiments, microporous network 300 extends all the way through microporous network 110. Of course, a microfluidic device may be constructed in any suitable fashion to achieve differential diffusion rates between microfluidic conduits and may have more than two different interconnected microporous networks and may have PDMS regions without pores to further reduce the rate of diffusion between selected conduits.

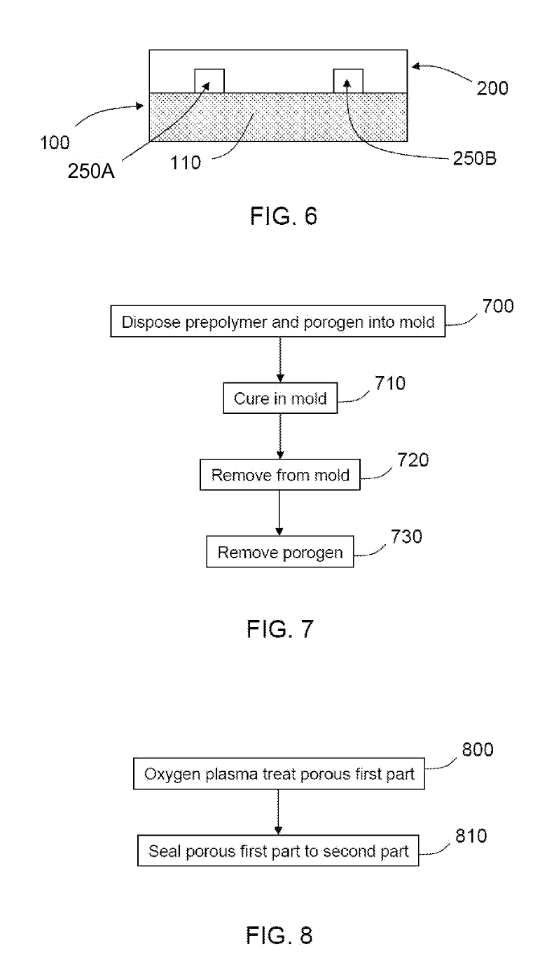

With reference now to FIG. 6, a microfluidic device may be formed of two PDMS parts 100, 200, where channels are formed in the non-porous PDMS part 200. The conduits 250A, 250B resulting from the channels are in fluid communication with the microporous network 110 of the microporous PDMS part, which can serve as the top or bottom walls of the conduits 250A, 250B.

The microfluidic devices described herein may be made by any suitable technique. PDMS may be made microporous through any suitable process. For example, PDMS may be extruded or molded with CO₂, may be foamed prior to extrusion or molded, or porogen may be introduced and then removed to render PDMS microporous. If a porogen is employed, the porogen may be blended or otherwise mixed with a PDMS prepolymer, such as Sylgard® 184 (Dow Corning Corporation, Midland, Mich., USA) and GE RTV 615 A+B kit (G. e. Silicones, Waterford, N.Y., USA). Non-limiting examples of porogens include salts, such as sodium bicarbonate, gelatin beads, sugar crystals, polymeric microparticles, and the like. One or more porogen may be incorporated into a preprolymer or polymer prior to curing or setting. The polymer may then be cured or set, and the porogen may be extracted with an appropriate solvent. In various embodiments, a porogen has an average diametric dimension of about 10 micrometers to about 1000 micrometers, about 50 micrometers to about 1000 micrometers, or about 100 micrometers to about 1000 micrometers.

The size and degree of porosity of PDMS material may be controlled by the size and concentration of porogen used, the extent of mixing with gas or foaming, etc. Accordingly, the rate at which a given gas may diffuse through the microporous PDMS may be controlled by varying the conditions under which pores are generated, as pore size and degree of porosity are related to diffusion rate.

In many embodiments, the porous PDMS part is molded using a master, such as a silicon master. The master may be fabricated from silicon by proximity UV photolithography. By way of example, a thin layer of photoresist, an organic polymer sensitive to ultraviolet light, may be spun onto a silicon wafer using a spin coater. The photoresist thickness is determined by the speed and duration of the spin coating. After soft baking the wafer to remove some solvent, the photoresist may be exposed to ultraviolet light through a photomask. The mask's function is to allow light to pass in certain areas and to impede it in others, thereby transferring the pattern of the photomask onto the underlying resist. The soluble photoresist is then washed off using a developer, leaving behind a protective pattern of cross-linked resist on the silicon. At this point, the resist is typically kept on the wafer to be used as the topographic template for molding the stamp. Alternatively, the unprotected silicon regions can be etched, and the photoresist stripped, leaving behind a wafer with patterned silicon making for a more stable template. If high resolution masters are desired, electron beam lithography on PMMA (polymethylmetacrylate). Templates can also be produced by micro machining, or they can be prefabricated by, e.g., diffraction gratings.

To enable simple demoulding of the master, an anti-adhesive treatment may be carried out using silanization in liquid phase with OTS (octadecyltrichlorosilane) or fluorinated silane, for example. After developing, the wafers may be vapor primed with fluorinated silane to assist in the subsequent removal of the array of projections. Examples of fluorinated silane that may be used include, but are not limited to, (tridecafluoro-1,1,2,2-tetrahydroctyl) trimethoxysilane, and tridecafluoro-1,1,2,2-tetrahydrooctyl) triethoxysilane.

In some embodiments, hot embossing or injection molding may be used to form the resulting polymer. However, the silicon master may not hold up well under conditions for such processes. In such cases, a reverse silicon master can be made and a metal, such as nickel, may be deposited on the reverse master to create a metal master for use in such processes.

In some embodiments, soft lithography may be employed and molds may be generated via any suitable technique, such as those described in P. K. Yuen and V. N. Goral, “Low-cost rapid prototyping of flexible microfluidic devices using a desktop digital craft cutter”, Lab on a Chip, 2010, 10, 384-387, where a digital desktop cutter is employer to form patterned structures out of films.

Regardless of the technique employed, the molds should have sufficient resolution to allow for generation of channels for forming the conduits of the device. In many embodiments, the channels and resulting conduits have a width of 0.1 mm or greater, and thus nearly any technique should be suitable.

If the device is a two or more part device, the optically transparent part or parts (e.g., the top part 200 in FIGS. 1-5) may be made of any suitable material capable of forming a seal or capable of being sealed with the microporous PDMS part. PDMS parts will readily self-seal with parts made from a number of materials, such as glass or other PDMS parts, particularly if a surface to be sealed is oxygen plasma treated. Otherwise, the parts may be adhered or the like to sealingly engage.

In embodiments where more than one microporous PDMS network is employed (see, e.g., items 300 and 110 in FIG. 5), the microporous networks, e.g. 110 and 300, may be fabricated separately and then bond together using oxygen plasma treatment. In this case, a well defined interface between network 110 and network 300 will be achieved. An alternative method would be during molding, prepolymer with porogen for network 300 will be added first and then prepolymer with porogen for network 110 will be added to the rest of the mold and on top of the prepolymer with porogen for network 300. The prepolymer may then be cured and porogens removed. Of course, any other suitable technique or process may be employed to fabricate devices having more than one PDMS microporous network.

While any suitable technique or process may be employed to form microfluidic devices or parts thereof, representative examples of overviews of methods that may be employed are shown in FIGS. 7-8. For purposes of convenience and clarity, the parts and features labeled in FIGS. 1-3 will be described with regard to the methods of FIGS. 7-8.

Referring now to FIG. 7, a composition that includes PDMS prepolymer and a porogen disposed in a mold (700). The mold is configured to form a first part 100 of the device 10 such that the first part 100 includes first and second channels 120. The composition is cured in the mold (710), resulting in a molded first part 100 having a microporous PDMS polymer with interspersed porogen. The first part 100 is removed from the mold (720) and the porogen is removed (730), e.g., by contacting with an appropriate solvent for the porogen. Of course the porogen may be removed from the molded first part prior to removing the part from the mold.

Referring now to FIG. 8, an overview of a method for sealing two parts of a microfluidic device is shown. The method includes providing the first 100 and second 200 parts. As used herein, “providing,” as it relates to a method, means to manufacture, purchase, or otherwise obtain. A surface of the first PDMS part 100 is oxygen plasma treated (800) and the first part is sealed to the second part 200 via self sealing, provided that the second part 200 is capable of self sealing with the PDMS first part 100. Of course, in some cases, a surface of the second part 200 may be oxygen plasma treated to facilitate self-sealing with the first PDMS part 100.

The microfluidic devices described herein may be used for any suitable purpose. For example, the devices may be advantageously employed in situations where interaction or exchange of gasses between conduits, but not interaction or exchange of aqueous liquids between conduits, is desired. For example, the microfluidic devices may be used for cell culture with the interconnected microporous network providing for rapid exchange of carbon dioxide and oxygen from and to cells. Cells and a cell culture medium may be introduced into one conduit, while a gaseous composition including oxygen may be introduced into another conduit. By way of further example, the microfluidic device may be used for a liquid-gas reaction, where an aqueous reagent is introduced into one conduit and a gaseous reagent is introduced into another conduit. Of course, the microfluidic devices described herein may be used for purposes of sample filtration, fluid mixing, valving, or the like.

In various aspects, methods and devices are described herein.

In a first aspect, a microfluidic device includes (i) a first conduit; (ii) a second conduit; and (iii) a first interconnected microporous network in communication with the first and second conduits and configured to allow diffusion of gas between the first and second conduits. The microporous network comprises PDMS and prevents flow of aqueous fluid between the first and second conduits through the microporous network.

A second aspect is a device according to the first aspect, wherein the interconnected microporous network has a water contact angle of 90 degrees or greater.

A third aspect is a device according to either of the first two aspects, wherein the interconnected microporous network comprises pores formed from porogens having an average particle size of between 10 micrometers and 1000 micrometers.

A fourth aspect is a device according to any of the preceding aspects, wherein at least a portion of the first and second conduits are formed in the material forming interconnected microporous network.

A fifth aspect is a device according to any of the preceding aspects, wherein the first and second conduits have a width of 0.1 millimeter or greater.

A sixth aspect is a microfluidic device according to any of the preceding aspects, further comprising: (i) a third conduit; and (ii) a second interconnected microporous network in communication with the second and third conduits and configured to allow diffusion of gas between the second and third conduits, wherein the second microporous network comprises PDMS and prevents flow of aqueous fluid between the second and third conduits through the second microporous network, wherein the average size of the pores of the first and second interconnected porous networks are different.

A seventh aspect is a device according to any of the preceding aspects, wherein at least a portion of the first conduit is formed from an optically transparent non-porous material.

An eight aspect is a microfluidic device according to any of the preceding aspects, wherein the device comprises: (i) a first part comprising the first interconnected microporous network; and (ii) an optically transparent second part, wherein the first and second parts together form the first and second conduits.

A ninth aspect is a micro fluidic device according to the eighth aspect, wherein the first part defines first and second channels and wherein a surface of the second part and the first and second channels together form the first and second conduits.

A tenth aspect is a micro fluidic device according to the eighth or ninth aspect, wherein the second part is a film.

An eleventh aspect is a microfluidic device according to the eighth or ninth aspect, wherein the second part is formed from non-porous PDMS.

A twelfth aspect is a method for culturing cells that includes: (i) inserting cells into the first conduit of a microfluidic device according to any of aspects 1-11; (ii) introducing cell culture medium into the first conduit to contact the cells; and (iii) flowing a gaseous composition comprising oxygen through the second conduit in the microfluidic device.

A thirteenth aspect is a method for reacting a gaseous reagent with an aqueous reagent that includes: (i) inserting a composition comprising the aqueous reagent into the first conduit of a microfluidic device according to any of aspects 1-11; (ii) introducing the gaseous reagent into the second conduit of the microfluidic device; and (iii) allowing the gaseous reagent to diffuse from the second conduit through the first interconnected microporous network to the first conduit to contact with the aqueous reagent.

A fourteenth aspect is a method for manufacturing a microfluidic device, which includes: (i) disposing a composition in a mold, the composition comprising a PDMS prepolymer and a porogen, the mold configured to form a first part of the microfluidic device, the part having first and second channels; (ii) curing the composition in the mold to form the first part, wherein the material forming the first part comprises a PDMS polymer interspersed with the porogen; (iii) removing the porogen from the PDMS polymer to generate a porous first part comprising a porous PDMS polymer; and (iv) sealing the porous first part to a second part such that a surface of the second part and the first and second channels of the first part together form first and second conduits of the microfluidic device.

A fifteenth aspect is a method according to the fourteenth aspect, wherein the porogen has an average particle size of between 10 micrometers and 1000 micrometers.

A sixteenth aspect is a method according to the fourteenth or fifteenth aspect, wherein the second part comprises an optically transparent portion configured to align with the first channel to allow viewing of the first conduit.

A seventeenth aspect is method according to the sixteenth aspect, wherein the second part is a film.

An eighteenth aspect is a method according to the sixteenth aspect, wherein the second part is formed from non-porous PDMS.

A nineteenth aspect is a method according to any of aspects 14-18, wherein a surface of the porous first part is oxygen plasma treated prior to sealing with the second part.

A twentieth aspect is a method for manufacturing a microfluidic device that includes: (i) disposing a composition in a mold, the composition comprising a PDMS prepolymer, the mold configured to form a first part of the microfluidic device, the part having first and second channels; (ii) curing the composition in the mold to form the first part, (iii) providing a second part of the device, the second part comprising PDMS having an interconnected microporous structure; and (iii) sealing the first part to a second part such that a surface of the second part and the first and second channels of the first part together form first and second conduits of the microfluidic device.

In the following, non-limiting examples are presented, which describe various embodiments of the articles and methods discussed above.

### EXAMPLES

### Example 1

### Device Design, Fabrication and Assembly

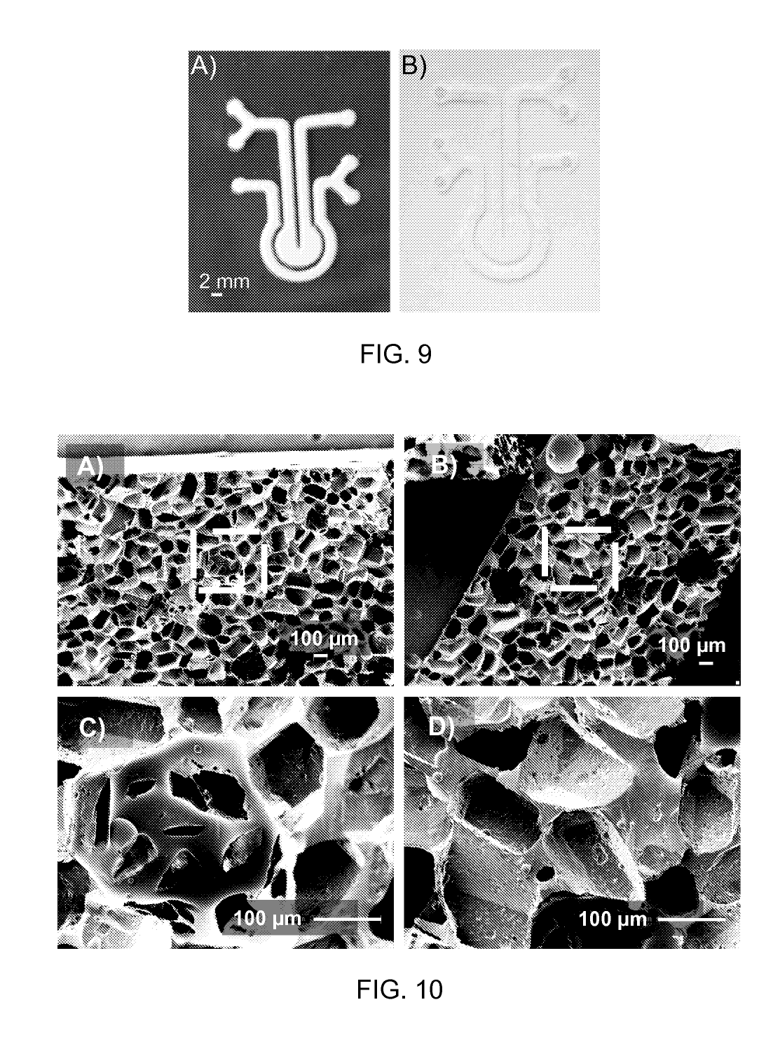

3D interconnected microporous PDMS microfluidic devices were developed to demonstrate the potential application of such devices via a gas absorption reaction. The device include of a 9.4 mm diameter inner circular chamber and a 2 mm wide outer circular channel separated by a 1 mm wide circular wall. Soft lithography was used to fabricate the microporous PDMS microfluidic device. Briefly, a microstructured mold was fabricated by stacking three custom cut 90 μm thick self-adhesive white vinyl sheets (Item # 699009; The Paper Studio®, Oklahoma City, Okla., USA) onto a glass slide (FIG. 9A). A PDMS prepolymer (10:1 w/w) (Sylgard® 184, Dow Corning Corporation, Midland, Mich., USA) mixed with pre-sieved sugar particles (1:2.5 v/v %) was cast onto the mold to a thickness of 2 mm and cured at 60° C. for overnight. The 2 mm thick microstructured PDMS replica was carefully peeled away. Then, the sugar particles in the microstructured PDMS replica were dissolved and washed away by soaking and washing in 20% ethanol solution in a ultrasonic cleaner (Model FS220H; Fisher Scientific, Pittsburgh, Pa., USA) for at least 3 hours before drying in air or in a 60° C. oven.

After the removal of sugar particles, the 3D interconnected microporous structures were formed in the microstructured PDMS replica (FIG. 10). Next, the microstructured microporous PDMS replica and a 2 mm thick non-porous PDMS replica, which was fabricated without any sugar particles, with inlet and outlet holes were treated with oxygen plasma in a RF plasma chamber (Model MPS-300; March Instruments, Inc., Concord, Calif., USA) at 60 W for 30 s before they were aligned, assembled together and incubated at 60° C. for overnight. After overnight incubation, the two replicas were irreversibly bonded together (FIG. 9B). Even though oxygen plasma treatment can change PDMS surface's wettability, the PDMS surface quickly returns to its hydrophobic behavior. The non-porous PDMS replica was used for ease of visualization. If desired, a microporous PDMS replica can be used to enclose the microstructured microporous PDMS replica so that the whole device can be fabricated from microporous PDMS. Also, microporous PDMS microfluidic devices in 3D configurations can be fabricated by repeating the above steps.

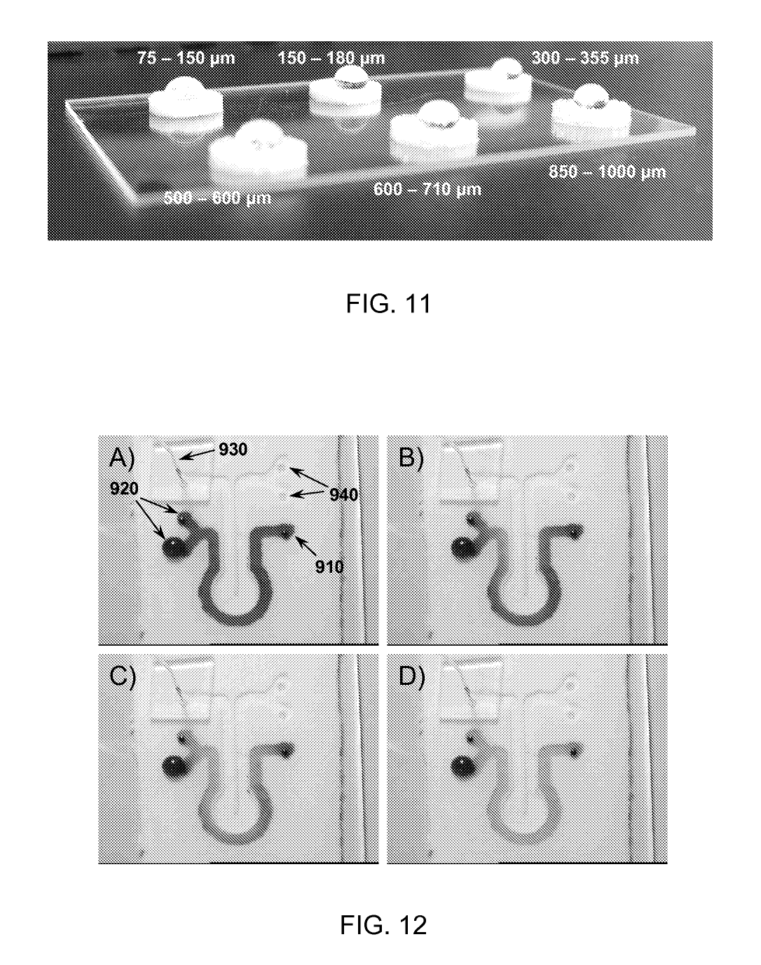

In order to demonstrate the pore size tunability and microporous PDMS surface wettability, pre-sieved sugar particles ranged from 75 μm to 1000 μm were used to fabricated various microporous PDMS structures (FIG. 11). The wettability study via a water droplet test indicated that hydrophobic behavior was exhibited on the surfaces of all the microporous PDMS structures tested by forming a near spherical droplet. Thus, 3D interconnected microporous PDMS microfluidic devices with tunable porosity can be fabricated by using different sizes of pre-sieved sugar particles.

The water contact angles of the resulting microporous PDMS was determined using an average of at least six locations for each sample. The results are presented below in Table 1.

TABLE 1
Water contact angle of porous PDMS

| Range of Pore Size | Water Contact |
|---|---|
| (micrometer) | Angle (°) |
| 75-100 | 97.35 |
| 150-180 | 112.66 |
| 300-355 | 116.43 |
| 500-600 | 124.73 |
| 600-710 | 92.01 |
| 850-1000 | 103.97 |

### Example 2

### Acidification of Water by CO₂ Gas

Bromothymol blue solution (Fluka® Analytical; Sigma-Aldrich® Corporation, St. Louis, Mo., USA) was used as a pH indicator solution to track the absorption of CO₂ gas by water. As water absorbs CO₂ gas, it reacts with the CO₂ gas to form carbonic acid. Thus, the pH indicator solution should turn from blue (pH>7.6) to green (pH˜6.5-7.0) and then to yellow (pH<6.0) depending on the amount of CO₂ gas was absorbed. The pH indicator solution with a pH of >7.6 (blue in color) was pipetted into the outer circular channel of the microporous PDMS microfluidic device (FIG. 12A) and was clearly retained inside the outer channel. The microporous structures remained dry during the experiment as indicated by their white appearance throughout the device (FIG. 12). Next, CO₂ gas was generated inside a glass bubbler by dissolving dry ice in water. As the CO₂ gas built up pressure inside the glass bubbler, it was directed through Tygon® tubing (Fisher Scientific, Pittsburgh, Pa., USA) into the inner circular chamber of the device without any other pumping means. This enabled CO₂ gas to gradually flow in and out of the inner chamber and to slowly diffuse into the outer channel through the microporous structures. It also prevented excess CO₂ gas from forcing the pH indicator solution out of the outer channel. The pH indicator solution slowly turned from blue to green and to yellow during the experiment and eventually, the whole outer channel turned yellow (FIG. 12). Also, the color of the pH indicator solution changed slowly along the inner wall of the outer channel following the CO₂ gas flow path. This indicated that CO₂ gas was gradually flowing in and out of the inner chamber, slowly diffusing through the microporous structures and was being absorbed by the liquid.

In FIG. 12, (A) is at time=0 sec.; (B) is at time=30 sec.; (C) is at time=50 sec.; and (D) is at time=90 sec. 910 indicates the inlet for pH indicator solution; 920 indicates the outlets for the pH indicator solution; 930 indicates the CO₂ gas inlet; and 940 indicated the CO₂ gas outlets.

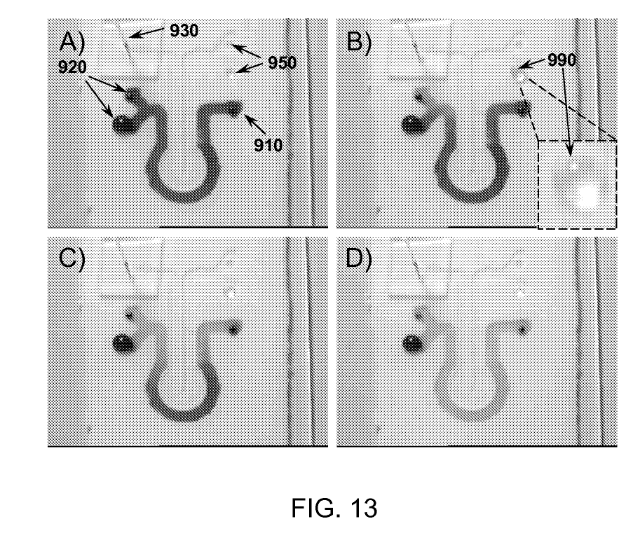

In a second CO₂ gas experiment, after pipetting the pH indicator solution into the outer circular channel of the microporous PDMS microfluidic device, the inner circular chamber was completely filled with sodium hydroxide solution before directing CO₂ gas into the inner chamber (FIG. 13A). If the sodium hydroxide solution leaked into the outer channel during the experiment, the pH indicator solution would remain blue or turn from yellow to blue (which was not observed). The sodium hydroxide solution also served to prevent CO₂ gas from flowing through the inner chamber. Thus, the path of CO₂ gas diffusion was through the microporous bottom device surface. Similar to the previous CO₂ gas experiment, the pH indicator solution slowly turned from blue to green and to yellow during the experiment indicating that CO₂ gas was slowly diffusing through the microporous bottom device surface (FIG. 13). However, in this case, the pattern of pH indicator solution color change was different (compared FIG. 12 with FIG. 13). As expected, the color change initiated at locations closest to the CO₂ gas inlet and slowly diffused outwards from the CO₂ gas inlet. Eventually, the whole outer channel turned yellow. In addition, gas bubbles 990 were occasionally observed at one of the sodium hydroxide solution outlets indicating that CO₂ gas was also being absorbed by the sodium hydroxide solution (insert in FIG. 13B).

In FIG. 13, (A) is at time=0 sec.; (B) is at time=45 sec.; (C) is at time=90 sec.; and (D) is at time=4 min. 30 sec. 910 indicates the inlet for pH indicator solution; 920 indicates the outlets for the pH indicator solution; 930 indicates the CO₂ gas inlet; and 950 indicated the sodium hydroxide solution outlets.

Thus, embodiments of MICROPOROUS MICROFLUIDIC DEVICE are disclosed. One skilled in the art will appreciate that the microfluidic devices and methods described herein can be practiced with embodiments other than those disclosed. The disclosed embodiments are presented for purposes of illustration and not limitation.

What is claimed is:

1. A micro fluidic device comprising:
a first conduit;
a second conduit; and
a first interconnected microporous network in communication with the first and second conduits and configured to allow diffusion of gas between the first and second conduits,
wherein the microporous network comprises poly(dimethylsiloxane) (PDMS) and prevents flow of aqueous fluid between the first and second conduits through the porous network.

2. A micro fluidic device according to claim 1, wherein the interconnected microporous network has a water contact angle of 90 degrees or greater.

3. A micro fluidic device according to claim 1, wherein the interconnected microporous network comprises pores formed from porogens having an average particle size of between 10 micrometers and 1000 micrometers.

4. A micro fluidic device according to claim 1, wherein at least a portion of the first and second conduits are formed in the material forming interconnected microporous network.

5. A micro fluidic device according to claim 1, wherein the first and second conduits have a width of 0.1 millimeter or greater.

6. A microfluidic device according to claim 1, further comprising:
a third conduit; and
a second interconnected microporous network in communication with the second and third conduits and configured to allow diffusion of gas between the second and third conduits, wherein the second microporous network comprises PDMS and prevents flow of aqueous fluid between the second and third conduits through the second microporous network,
wherein the average size of the pores of the first and second interconnected porous networks are different.

7. A micro fluidic device according to claim 1, wherein at least a portion of the first conduit is formed from an optically transparent non-porous material.

8. A micro fluidic device according to claim 1, wherein the device comprises:
a first part comprising the first interconnected microporous network; and
an optically transparent second part,
wherein the first and second parts together form the first and second conduits.

9. A microfluidic device according to claim 8, wherein the first part defines first and second channels and wherein a surface of the second part and the first and second channels together form the first and second conduits.

10. A micro fluidic device according to claim 8, wherein the second part is a film.

11. A micro fluidic device according to claim 8, wherein the second part is formed from non-porous PDMS or glass.

12. A method for culturing cells comprising:
inserting cells into the first conduit of a microfluidic device according to claim 1;
introducing cell culture medium into the first conduit to contact the cells; and
flowing a gaseous composition comprising oxygen through the second conduit the microfluidic device.

13. A method for reacting a gaseous reagent with an aqueous reagent, comprising:
inserting a composition comprising the aqueous reagent into the first conduit of a microfluidic device according to claim 1; and
introducing the gaseous reagent into the second conduit of the microfluidic device; and
allowing the gaseous reagent to diffuse from the second conduit through the first interconnected microporous network to the first conduit to contact with the aqueous reagent.

14. A method for manufacturing a micro fluidic device comprising:
disposing a composition in a mold, the composition comprising a PDMS prepolymer and a porogen, the mold configured to form a first part of the microfluidic device, the part having first and second channels;
curing the composition in the mold to form the first part, wherein the material forming the first part comprises a PDMS polymer interspersed with the porogen;
removing the porogen from the PDMS polymer to generate a porous first part comprising a microporous PDMS polymer; and
sealing the porous first part to a second part such that a surface of the second part and the first and second channels of the first part together form first and second conduits of the microfluidic device.

15. A method according to claim 14, wherein the porogen has an average particle size of between 10 micrometers and 1000 micrometers.

16. A method according to claim 14, wherein the second part comprises an optically transparent portion configured to align with the first channel to allow viewing of the first conduit.

17. A method according to claim 16, wherein the second part is a film.

18. A method according to claim 16, wherein the second part is formed from non-porous PDMS or glass.

19. A method according to claim 14, wherein a surface of the microporous first part is oxygen plasma treated prior to sealing with the second part.

20. A method for manufacturing a micro fluidic device comprising:
disposing a composition in a mold, the composition comprising a PDMS prepolymer, the mold configured to form a first part of the microfluidic device, the part having first and second channels;
curing the composition in the mold to form the first part;
providing a second part of the device, the second part comprising PDMS having an interconnected microporous structure; and
sealing the first part to a second part such that a surface of the second part and the first and second channels of the first part together form first and second conduits of the microfluidic device.
